(19) United States
(12) Patent Application Publication
(10) Pub. No.: US 2011/0040317 A1
(43) Pub. Date: Feb. 17, 2011

(54) LANCET BLOCK AND LANCET ACTIVATING DEVICE

(75) Inventors: Seung Seob Lee, Daejeon (KR); Boo Joon Sul, Daejeon (KR); Man Hee Han, Daejeon (KR)

Correspondence Address: Edwards Angell Palmer & Dodge LLP, P.O. Box 55874, Boston, MA, 02205, US

(73) Assignee: MITI SYSTEMS INC., Daejeon (KR)

(21) Appl. No.: 12/894,593

(22) Filed: Sep. 30, 2010

Related U.S. Application Data
(63) Continuation of application No. PCT/KR2010/003330, filed on May 26, 2010.

(30) Foreign Application Priority Data
Aug. 13, 2009 (KR) 10-2009-0074550

(51) Int. Cl. A61B 17/34

(52) U.S. Cl. 606/182

(57) ABSTRACT

A lancet block comprises a lancet comprising a cam projection and a lancet housing receiving the lancet in such a fashion that the lancet can be vertically moved relatively with respect to the lancet housing. A top wall of the lancet housing and a side wall of the lancet housing corresponding to the cam projection of the lancet are formed of a thin membrane made of a material capable of being easily disrupted and a bottom wall of the lancet housing is formed of a thin membrane made of a material capable of being easily disrupted by a tip of the needle of the lancet. With this lancet block, it is possible to, e.g., provide convenient and sanitary use of a lancet, avoid a risk of any injury by a needle tip, prevent bacterial infection due to such injury, and reduce manufacturing costs.

### CROSS-REFERENCE TO RELATED APPLICATION

This is a continuation of International Application No. PCT/KR2010/003330, with an international filing date of May 26, 2010, which claims the benefit of Korean Application No. 10-2009-0074550 filed Aug. 13, 2009, the entire contents of which are incorporated herein by reference.

### TECHNICAL FIELD

The present invention relates to a needle for micro blood sampling and a device equipped with the needle for application to the skin of a subject in need.

### BACKGROUND ART

A blood test is one of the most popular and accurate tests in medical diagnosis. Since several milliliters of blood is required to perform a variety of tests, the blood is taken from an artery or a vein using a syringe and the like. But several micro-liters of blood is enough in order to test only a single item such as blood glucose level.

Specifically, in the case of diabetes, since the blood sampling may be needed more than five times daily, it is important to increase convenience of use and minimize cost while minimizing pain felt by a user. To this end, a lancet and a lancet activating device are usually used.

In general, a lancet is constructed such that a metal needle is surrounded by a plastic housing. A needle tip is also surroundingly enclosed and shielded by a needle tip protection cover for aseptic purpose. The lancet is mounted to the lancet activating device and the needle tip protection cover is removed from the needle tip of the lancet in use. The lancet activating device is one which allows the needle tip of the lancet to be sequentially advanced and retracted rapidly

In the meantime, the lancet must be used on a disposable basis to prevent bacterial infection or hemic disease infection. However, since the lancet must be replaced with new one each time after use, its use is complicated. According to the circumstances, there often occurs the case where the lancet is re-used many times without replacement after use. Also, upon the replacement of the used lancet, a needle of the lancet is exposed outwardly from an opening of the lancet, and thus a user's finger may be pricked with a tip of the needle inadvertently. In addition, a worker who disposes of discarded lancets is also exposed to a risk of injury.

In order to address and solve such problems, European Patent No. EP 0 565 970 and U.S. Pat. No. 4,442,836 teach a lancet activating device which automatically ejects a used lancet. However, such a lancet activating device entails a problem in that since a needle tip of the ejected lancet is not protected, there is a risk of injury by the needle tip. Meantime, European Patent No. EP 0 595 148 and U.S. Pat. No. 5,454,828 disclose a lancet unit of a type in which a lancet is protected by a movable sleeve or an end cap. European Patent No. EP 0 630 609 and German Patent No. DE 103 12 357 propose a lancet activating device of a type in which a lancet is configured to inhibit any re-use of any component of the lancet activating device so as to prevent a careless user from re-inserting and using a needle that has been already ejected.

However, such conventional lancets and lancet activating devices involve drawbacks in that they are complicated in their constructions, which leads to an increase in the manufacturing cost, thereby decreasing economic efficiency, and in that a work of mounting a new lancet is complex, thereby deteriorating convenience of use.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention has been made in order to solve the above-described problems occurring in the prior art, and it is an object of the present invention to provide a lancet block and a lancet activating device, in which a plurality of lancets can be mounted at a time to allow a user to conveniently use them one by one.

Another object of the present invention is to provide a lancet block and a lancet activating device, in which an ejected needle tip of a lancet can be prevented from being exposed externally so as to reduce a risk of injury by the needle tip.

Yet another object of the present invention is to provide to a lancet block and a lancet activating device, in which a used lancet can be automatically ejected so as to prevent a user from re-using the used lancet.

Still another object of the present invention is to provide a lancet block which is deformed to prevent the lancet from being re-mounted during the use/ejection of the lancet, so that the re-use of the lancet can be prevented, and a lancet activating device.

To achieve the above objects, in one aspect, the present invention provides a lancet block including a lancet and a lancet housing for receiving the lancet therein.

The lancet comprises a body, a needle formed at the bottom of the body, and a cam projection of a predetermined length formed at a side of the body.

The lancet housing receives the lancet in such a fashion that the lancet can be vertically moved relatively with respect to the lancet housing and includes a stop projection formed at an outer side thereof.

A top wall of the lancet housing and a side wall of the lancet housing corresponding to the cam projection of the lancet are formed of a thin membrane made of a material capable of being easily disrupted or a material capable of being expanded elastically upon relative downward movement of the lancet with respect to the lancet housing. A bottom wall of the lancet housing is formed of a thin membrane made of a material capable of being easily disrupted by a tip of the needle of the lancet

In another aspect, the present invention provides a lancet activating device adapted to activate the above-described lancet block.

The lancet activating device comprises a lancet-receiving member, a rotation drive unit, a housing, a load button, a vertical return spring, and a rotational return spring.

The lancet-receiving member includes an upper portion and a lower portion extending by a predetermined length from a lower distal end thereof, the lower portion having an outer diameter smaller than that of the upper portion. The lancet-receiving member includes a lancet block movement passageway penetratingly formed longitudinally therein so that one or more lancet blocks can be fittingly inserted into the lancet block movement passageway in the longitudinal direction so as to be vertically and longitudinally moved but restricted in their rotation along the lancet block movement passageway and a cam projection exposure slot opening formed longitudinally on the periphery of the lower portion of the lancet-receiving member so as to allow a cam projection of a lancet block to be exposed externally therethrough. The lancet-receiving member is fittingly inserted into the rotation drive unit in such a fashion that an outer periphery of the lancet-receiving member comes into close contact with an inner periphery of the rotation drive unit so that the lancet-receiving member can rotate relatively with respect to the rotation drive unit within the rotation drive unit, and is fixedly coupled to the housing.

The rotation drive unit is configured such that the lancet-receiving member is fittingly inserted into the rotation drive unit in such a fashion that the outer periphery of the lancet-receiving member comes into close contact with the inner periphery of the rotation drive unit. The load button is fittingly inserted into an upper end of the rotation drive unit in such a fashion that the inner periphery of the load button comes into close contact with the outer periphery of the rotation drive unit so that the load button can move vertically in the longitudinal direction thereof. The rotation drive unit has a cam projection guide slot formed on the periphery of a lower end portion thereof to correspond to the cam projection exposure slot opening of the lancet-receiving member so that the cam projection of the lancet block protruded outwardly through the cam projection exposure slot opening is fittingly inserted into the cam projection guide slot so as to allow the lancet to be guided to move in the direction of downwards (for its skin penetration), upwards (for its separation from the skin) and downwards (for its ejection) in this order, a retaining step circumferentially formed on one side of the inner peripheral surface thereof so as to temporarily restrict the downward movement of the stop projection of the lancet block, an elastic load-maintaining lug formed on the outer peripheral surface of a lower portion thereof in such a fashion as to be spaced remote from the cam projection guide slot, and at least one support jaw formed on the outer peripheral surface thereof so as to be received in one or more support barrier walls of the housing so that the longitudinal movement of the rotation drive unit is restricted and the rotation of the rotation drive unit is enabled in response to the vertical movement of the load button;

The housing includes one or more support barrier walls formed on the inner peripheral surface thereof so as to receive the support jaw of the rotation drive units in a space defined between the support barrier walls so that the rotation drive unit is mounted within the housing in such a fashion that the longitudinal movement of the rotation drive units is restricted and the rotation of the rotation drive units is enabled. The housing is fixedly coupled with the lancet-receiving member. The load button is fittingly inserted into an upper end of the housing in such a fashion that the outer periphery of the load button comes into close contact with the inner periphery of the housing so that the load button is restricted in the rotation thereof and can move vertically in the longitudinal direction thereof. The housing has a through-hole formed on the outer peripheral surface thereof to correspond to the load position of the load-maintaining lug of the rotation drive unit, and a drive button 51 fittingly mounted into the through-hole so as to elastically apply a pressure to the load-maintaining lug when the drive button is pressed.

The load button is fittingly inserted between the upper end of the housing and the upper end of the rotation drive unit so that the load button can move only in the longitudinal direction thereof and allows the rotation drive unit to rotate upon the longitudinal movement thereof.

The vertical return spring is interposed between a support barrier wall of the housing and an open end of the load button so as to return the load button pushed downwardly by an external force to it is original position, and the rotational return spring is interposed between a support barrier wall of the housing and the support jaw of the rotation drive unit in such a fashion as to be fixedly secured at one end to the housing and at other end to the rotation drive unit so as to return the rotation drive unit rotated by means of an external force to its original position before the rotation.

In addition, according to the present invention, when a lancet is mounted within the lancet activating device or after the lancet is used, a tip of the needle 13 of the lancet is prevented from being exposed externally, thereby avoiding a risk of any injury by the needle tip and preventing bacterial infection due to such injury.

Besides, according to the present invention, a tip of the lancet needle is formed of a polymeric material so that the manufacturing process is simplified, thereby reducing the manufacturing cost.

The above and other features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIG. 1 is a perspective view illustrating a lancet (A) and a lancet housing (B) according to the present invention;

FIG. 2 is a cross-sectional perspective view illustrating a lancet block in which a lancet and a lancet housing are assembled together according to the present invention;

FIG. 3 is a perspective view illustrating a lancet housing additionally including a re-use prevention member before (A) and after (B) of the use of the lancet housing;

FIG. 4 is a perspective view illustrating a lancet-receiving member as a constituent element of a lancet activating device according to the present invention;

FIG. 5 is a perspective view illustrating a rotation drive unit as a constituent element of a lancet activating device according to the present invention;

FIG. 6 is a perspective view illustrating a lower end portion of the rotation drive unit having a cam projection guide slot formed on the periphery thereof;

FIG. 7 is an expanded plan view illustrating the lower end portion of the rotation drive unit having a cam projection guide slot formed on the periphery thereof;

FIG. 8 is a cross-sectional perspective view illustrating the lower end portion of the rotation drive unit having a retaining step circumferentially formed on one side of the inner peripheral surface thereof;

FIGS. 9A and 9B are perspective and cross-sectional views illustrating a housing as a constituent element of a lancet activating device according to the present invention;

FIG. 10 is a perspective view illustrating a load button as a constituent element of a lancet activating device according to the present invention;

FIG. 11 is a cross-sectional view illustrating a lancet activating device according to the present invention in which all the constituent elements are assembled together;

FIG. 12 is a perspective view illustrating a series of operation processes of a load button of a lancet activating device according to the present invention;

FIG. 13 is a view illustrating a series of operation processes of a cam projection inserted into a cam projection guide slot of a rotation drive unit and a lancet block upon the reverse rotation (A) and forward rotation (B) of a lancet activating device;

FIG. 14 is a partial cross-sectional view illustrating a state in which a cam projection is positioned at the lowermost vertex of a cam projection guide slot of a rotation drive unit upon the reverse rotation of a lancet activating device; and

FIG. 15 is a cross-sectional perspective view illustrating a lancet block finally ejected from a lancet activating device after its use.

### DETAILED DESCRIPTION OF THE DISCLOSURE

Hereinafter, the present invention will be described in detail in connection with the preferred embodiments with reference to the accompanying drawings. However, these embodiments are for illustrative purposes, and the scope of the present invention is not limited thereto. Also, it will be understood by those skilled in the art that various modifications and variations can be made to the present invention without departing from the spirit and scope of the appended claims based on the illustrative embodiments.

In the drawings, although it has been shown that a lancet body 11, a lancet housing 20 and a lancet block 100 are formed in a cubic shape, this is merely illustrative and they may be formed in other shapes (e.g., a cylindrical shape).

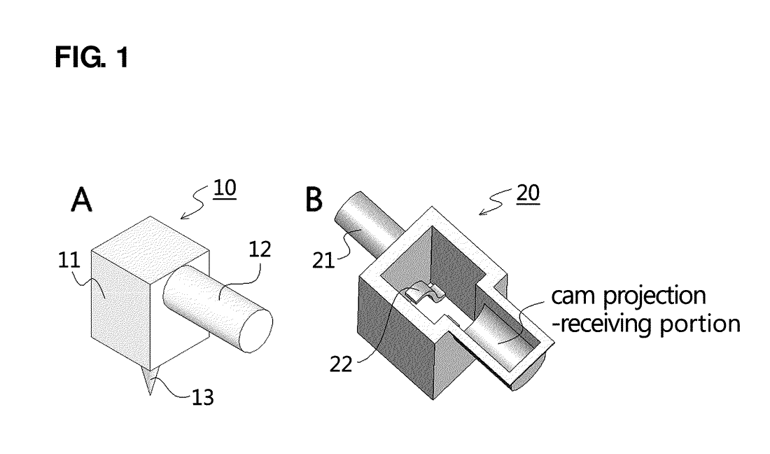

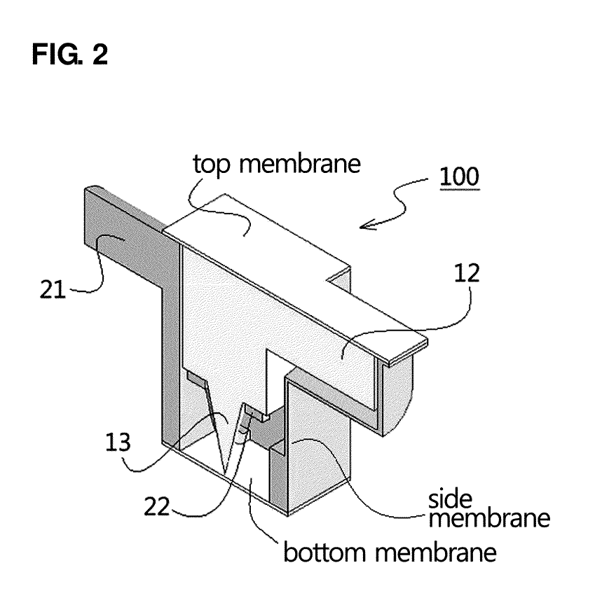

FIG. 1 is a perspective view illustrating examples of a lancet (A) and a lancet housing (B) according to the present invention, and FIG. 2 is a cross-sectional perspective view illustrating an example of a lancet block 100 in which a lancet 10 and a lancet housing 20 are assembled together according to the present invention. For reference, for the sake of a clearer understanding of the present invention, in the drawings, the shapes and sizes of respective elements may be exaggerated for clarity.

In the present invention, the lancet 10 is constructed to include a cubic-shaped body 11, a needle 13 formed at the bottom of the body 11, and a cam projection 12 of a predetermined length formed at a side of the body 11. The outer diameter of the cam projection 12 corresponds to the width of a cam projection exposure slot opening 32 formed on a lancet-receiving member 30 and the width of a cam projection guide slot 42 formed on a rotation drive unit. The cam projection 12 is sized in length so as to be fittingly inserted into the projection guide slot 42 of the rotation drive unit. That is, the length of the cam projection 12 is enough if the cam projection 12 is protruded outwardly and is retained in the cam projection guide slot 42 of the rotation drive unit 40 when passing through the cam projection exposure slot opening 32 of the lancet-receiving member 30.

The lancet 10 may be manufactured in such a fashion that a metal needle is surrounded by a plastic housing similar to a conventional lancet. It is easier to form the lancet 10 with a biocompatible thermoplastic resin using a mold with a negative pattern of a needle during the injection-molding process. In order to mold the needle with a plastic material, it is preferable to use a super-engineering plastic material such as polyetheretherketone (PEEK), polyetherimide (PEI) or the like having a high strength and biocompatibility, rather than a general-purpose plastic material having a low strength, i.e., acrylonitrile butadiene styrene (ABS), polymethyl methacrylate (PMMA) or the like.

In the meantime, although the above-constructed lancet is packed in a sterilized state, since it is exposed to the atmosphere during its use, it may be difficult to maintain the lancet in a complete sterilized state before its use. In order to overcome the contamination problems of the lancet, the present invention provides a lancet block in which one lancet 10 is mounted within one lancet housing 20.

In the present invention, the lancet housing 20 is configured such that the lancet 10 is mounted within the lancet housing in such a fashion as to be able to be vertically moved with respect to the lancet housing. The shape of the lancet housing 20 is not limited. Examples of the shape include a polygonal or cylindrical box. However, since the lancet housing 20 is eventually fittingly inserted into and can be moved along a lancet block movement passageway 33 formed in the lancet-receiving member 30 in the longitudinal direction thereof, the transversal cross-sectional shape of the lancet housing must correspond or substantially correspond to the transversal cross-sectional shape of the lancet block movement passageway 33. They, however, are not required to be identical.

When the lancet activating device is actuated, there is a time point when the lancet housing 20 is temporarily stopped in its vertical movement and only the lancet mounted within the lancet housing is vertically moved, which will be described later. For this step, the stop projection 21 is formed at an outer side of the lancet housing 20. The position and size of the stop projection 21, however, may be selected properly depending on the circumstances.

A top wall of the lancet housing 20 and a side wall of the lancet housing 20 where a cam projection-receiving portion for receiving the cam projection 12 of the lancet 10 therein is formed are formed of a thin membrane made of a material capable of being easily disrupted or a material capable of being expanded elastically upon the relative downward movement of the lancet 10 with respect to the lancet housing, and a bottom wall of the lancet housing 20 is formed of a thin membrane made of a material capable of being easily disrupted by a tip of the needle 13 of the lancet 10. In the case where a side wall of the lancet housing 20 is formed of an elastic material, when a pressure causing the relative downward movement of the lancet 10 with respect to the lancet housing is released, the lancet returns to its original position by an elastic restoring force. In the case where the side wall of the lancet housing is not formed of an elastic material, a spring 22 is preferably further provided within the lancet housing 20 so as to return the lancet 10 moved downwardly relatively with respect to the lancet housing to its original position.

According to the present invention, since the lancet stays inside the lancet housing 20 until it is applied to the skin of a patient, it can be maintained in a sterilized state.

Meanwhile, the lancet housing 20 may be further provided with a certain structure so as to prevent a careless user from re-mounting and using an ejected lancet. That is, the lancet housing 20 preferably further includes a re-use prevention member 23 formed on the bottom of the stop projection 21 thereof so as to allow the re-use prevention member to be disrupted or deformed by a pressure, making it impossible for the lancet housing to be re-inserted into the lancet-receiving member 30.

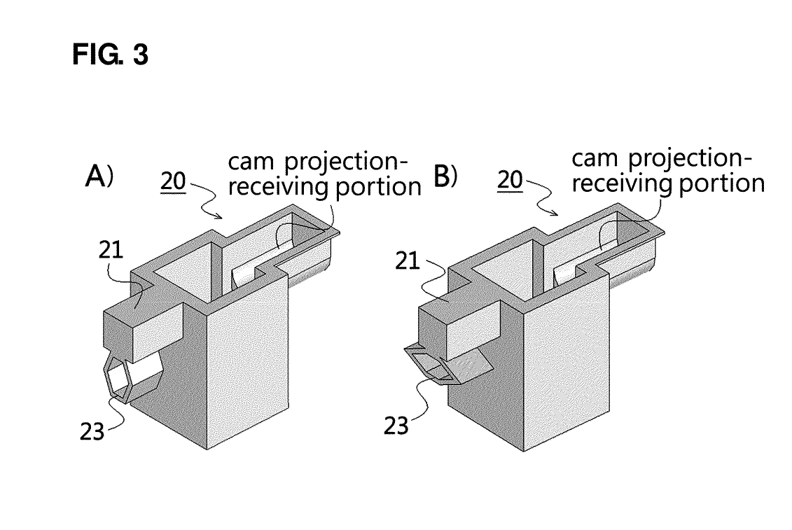

FIG. 3 is a perspective view illustrating a lancet housing additionally including the re-use prevention member 23 before (A) and after (B) of the use of the lancet housing.

When the lancet block passes through the lowermost point, the re-use prevention member 23 is deformably folded by a retaining step 45 of cylindrical rotation drive unit 40 to cause the cross-sectional area of the re-use prevention member 23 to be increased, making it impossible for the lancet block to be inserted into the lancet-receiving member 30. Although a ring-shaped re-use prevention member 23 is shown in FIG. 3, the re-use prevention member may be formed in various shapes such as ‘ [symbol] ’-shape and the like. In addition, the re-use prevention member 23 may be deformed in a foldable manner, and may be deformed in such a fashion as to be partially torn or broken to cause the cross-sectional area thereof to be increased. Furthermore, the re-use prevention member 23 may be formed on the bottom surface of the stop projection 21, and the deformation of the cam projection-receiving portion receiving the cam projection 12 of the lancet 10 or its surrounding or peripheral portion may be used.

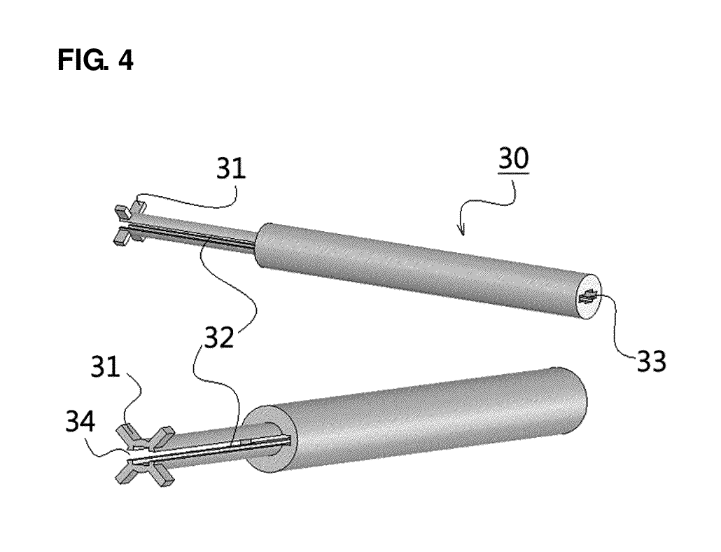

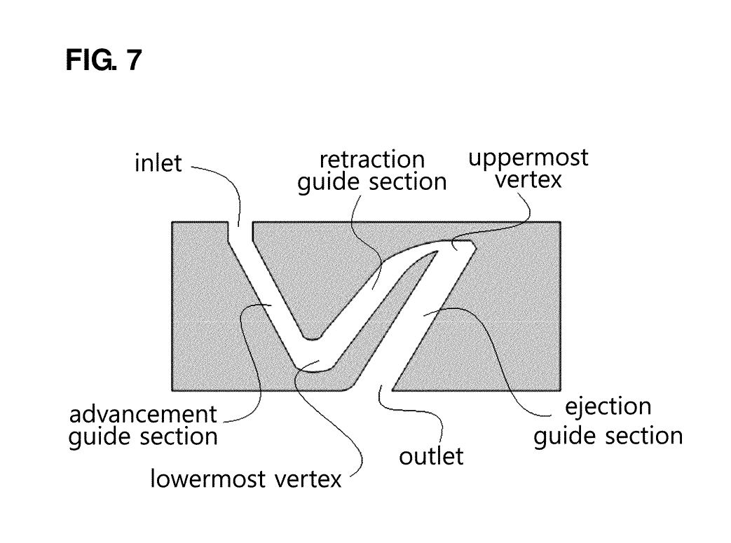

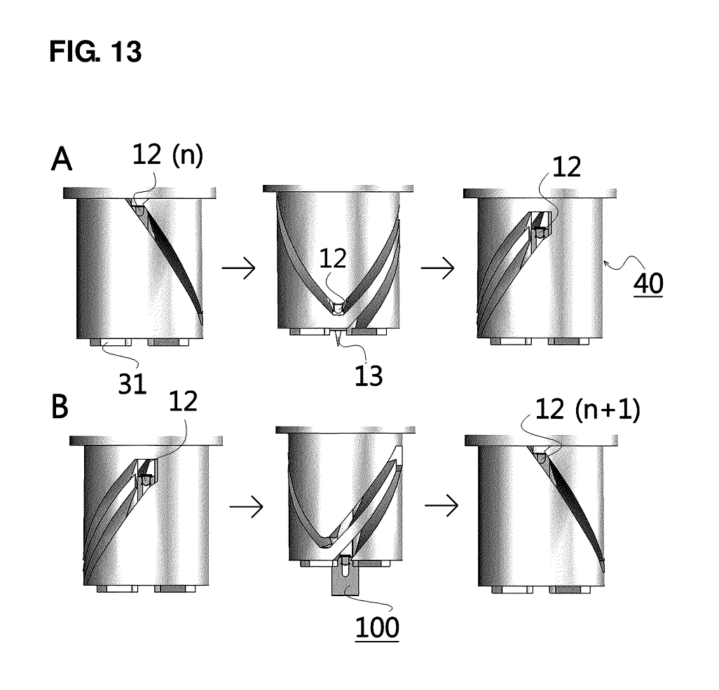

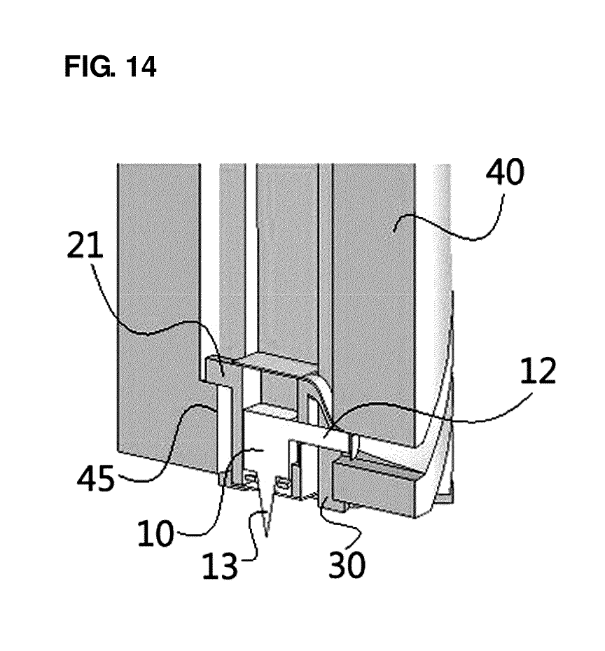

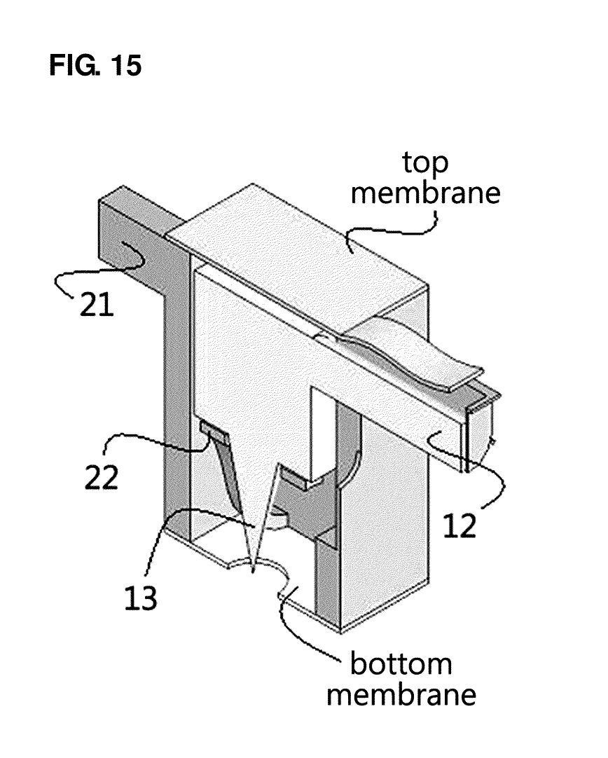

The present invention will be described with reference to FIGS. 4-15.

In the present invention, the lancet-receiving member is fittingly inserted into the rotation drive unit in a state where one or more lancet blocks are received in the lancet-receiving member. To this end, the lancet-receiving member 30 includes a lancet block movement passageway 33 penetratingly formed longitudinally therein so that one or more lancet blocks can be fittingly inserted into the lancet block movement passageway in the longitudinal direction so as to be vertically movable but restricted in their rotation along the lancet block movement passageway. Also, as shown in FIG. 4, the lancet-receiving member 30 includes an upper portion and a lower portion extending by a predetermined length from a lower distal end thereof, the lower portion having an outer diameter smaller than that of the upper portion, so that a cam projection exposure slot opening 32 and a stop projection exposure slot opening 34 are formed longitudinally on the periphery of the lower portion of the lancet-receiving member so as to allow the cam projection 12 and the stop projection 21 of a lancet block to be exposed externally therethrough, respectively. Although it is shown in FIG. 4 that both the cam projection exposure slot opening 32 and the stop projection exposure slot opening 34 are formed, the stop projection exposure slot opening 34 does not necessarily need to be formed. The lancet-receiving member 30 is fittingly inserted into the cylindrical rotation drive unit with an outer periphery of the lancet-receiving member coming into close contact with an inner periphery of the cylindrical rotation drive unit so that the lancet-receiving member can rotate relatively with respect to the rotation drive unit within the rotation drive unit 40. Also, the lancet-receiving member 30 is fixedly coupled to a cylindrical housing 50. The type and location of a coupling portion between the lancet-receiving member 30 and the cylindrical housing 50 are not limited to particular ones, although it is shown in the drawings that lancet-receiving member 30 is coupled to the housing 50 by means of coupling lugs 31 formed at a distal end of the lower portion of the lancet-receiving member 30.

As mentioned above, since the lancet housing 20 must be eventually fittingly inserted into and moved along a lancet block movement passageway 33 formed in the lancet-receiving member 30 in the longitudinal direction thereof, a transversal cross-sectional shape of the lancet housing must correspond to a transversal cross-sectional shape of the lancet block movement passageway 33, but they are not required to be identical. That is, any cross-sectional shape of the lancet block movement passageway 33 can be used as long as the cam projection 12 of the lancet 10 inserted into the lancet block movement passageway is prevented from escaping from the lancet block movement passageway.

In the meantime, preferably, a cap mounted with a spring may be provided at a distal end of the upper portion of the lancet-receiving member so that the spring downwardly pushes the lancet blocks mounted inside the lancet-receiving member.

In the present invention, the rotation drive unit 40 serves to allow the lancet block to be vertically moved in the lancet-receiving member so that the needle 13 of the lancet block is protruded externally and returns to its original internal position while the rotation drive unit rotates and returns to its original position before rotation relatively with respect to the lancet-receiving member, and then allow the used lancet block to be ejected outwardly from the lancet-receiving member.

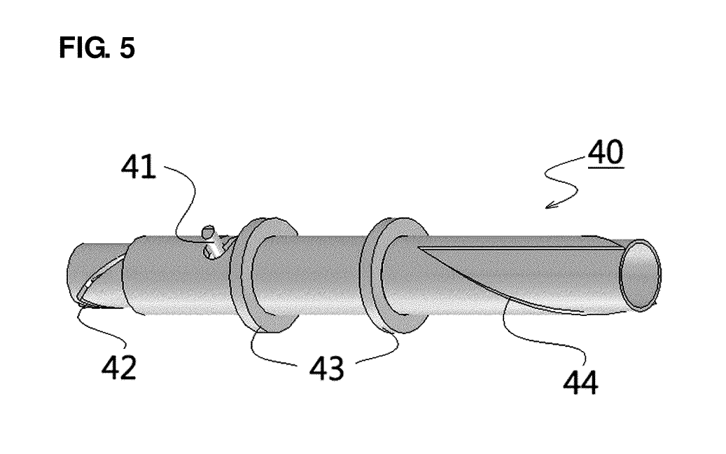

A load button 60 is fitted around an upper end portion of the rotation drive unit 40 in such a fashion that the inner periphery of the load button 60 comes into close contact with the outer periphery of the rotation drive unit 40 so that the load button can move vertically. The rotation drive unit 40 has a spiral rotation guide slot 44 formed on the upper end portion thereof. In this case, the rotation guide slot 44 may be formed on the outer surface of rotation drive unit 40 or may formed in an incised manner of the rotation drive unit 40 as shown in FIG. 5.

The rotation drive unit 40 has an elastic load-maintaining lug 41 formed thereon in such a fashion as to be positioned between the cam projection guide slot 42 and the spiral rotation guide slot 44. Also, the rotation drive unit 40 has a pair of opposed annular support jaws 43 formed on the outer peripheral surface thereof so as to allow the support jaws 43 to be received in a space defined between support barrier walls 54 formed on the inner peripheral surface of the cylindrical housing 50 so that the rotation drive unit is mounted within the cylindrical housing in such a fashion that the longitudinal movement of the rotation drive unit 40 is restricted and the rotation thereof is enabled. Although two support jaws 43 are shown in FIG. 5, the number and position of the support jaws may vary.

The lancet-receiving member is fittingly inserted into the cylindrical rotation drive unit 40 with an outer periphery of the lancet-receiving member coming into close contact with an inner periphery of the cylindrical rotation drive unit so that the lancet-receiving member can rotate relatively with respect to the rotation drive unit within the rotation drive unit. Since the outer diameter of the lower distal end of the lancet-receiving member is smaller than that of the upper portion thereof, the inner diameter of the lower distal end of the rotation drive unit 40 is smaller than that of the upper portion thereof.

The cylindrical rotation drive unit 40 includes a cam projection guide slot 42 formed on the periphery of a lower end portion thereof to correspond to the cam projection exposure slot opening 32 of the lancet-receiving member 30 so that the cam projection 12 of the lancet block protruded outwardly through the cam projection exposure slot opening 32 is fittingly inserted into the cam projection guide slot 42 so as to allow the lancet to be guided to move in the direction of downwards (for its skin penetration), upwards (for its separation from the skin) and downwards (for its ejection) in this order, in response to the rotation and returning of the rotation drive unit 40. The cam projection guide slot 42 may be penetratingly formed on the periphery of the rotation drive unit 40 or a cam projection guide groove may be formed on the inner peripheral surface of the rotation drive unit 40 as shown in FIG. 5.

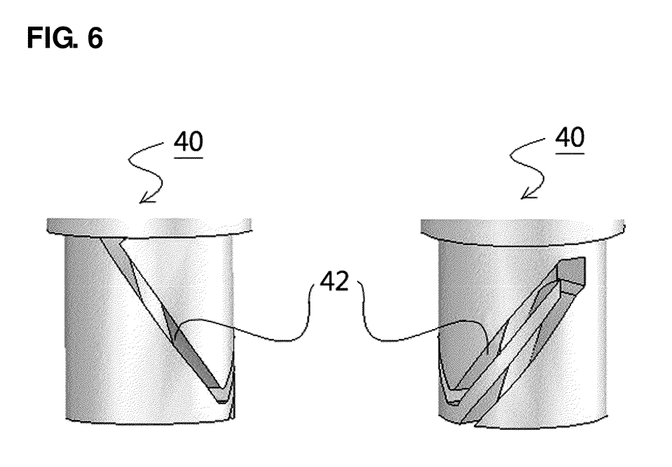

The cam projection guide slot 42 is formed to have a width enough for the cam projection 12 of the lancet block to be movable therealong and extends from an inlet, via a lowermost vertex and then an uppermost vertex, to an outlet. The inlet of the cam projection guide slot 42 is formed at a position corresponding to an upper end of the cam projection exposure slot opening 32 of the lancet-receiving member, and the outlet of the cam projection guide slot 42 is opened toward a lower end of the cylindrical housing 50 (see FIGS. 5 and 6). A guide slot section running from the inlet via the lowermost vertex to the uppermost vertex is formed in a “V” shape, and a guide slot section running from the uppermost vertex to the outlet is formed in an oblique shape.

In the cam projection guide slot 42, an inclined wall defined between a guide slot section running from the lowermost vertex to the uppermost vertex and a guide slot section running from the uppermost vertex to the outlet is made of an elastically deformable material, and a width of a guide slot section adjacent to the uppermost vertex is preferably formed smaller than the outer diameter of the cam projection 12. This structure of the cam projection guide slot 42 is intended to prevent a lancet block used immediately before insertion of a new lancet block into the lancet-receiving member 30 from being re-moved upwardly and facilitate ejection of the used lancet block from the lower distal end of the lancet-receiving member 30, which will be described later.

For the sake of convenience of explanation, a guide slot section running from the inlet to the lowermost vertex is referred to as an “advancement guide section”, a guide slot section running from the lowermost vertex to the uppermost vertex is referred to as a “retraction guide section”, a guide slot section running from the uppermost vertex to the outlet is referred to as an “ejection guide section”, and the inclined wall defined between the retraction guide section and the ejection guide section is referred to as an “elastic support”.

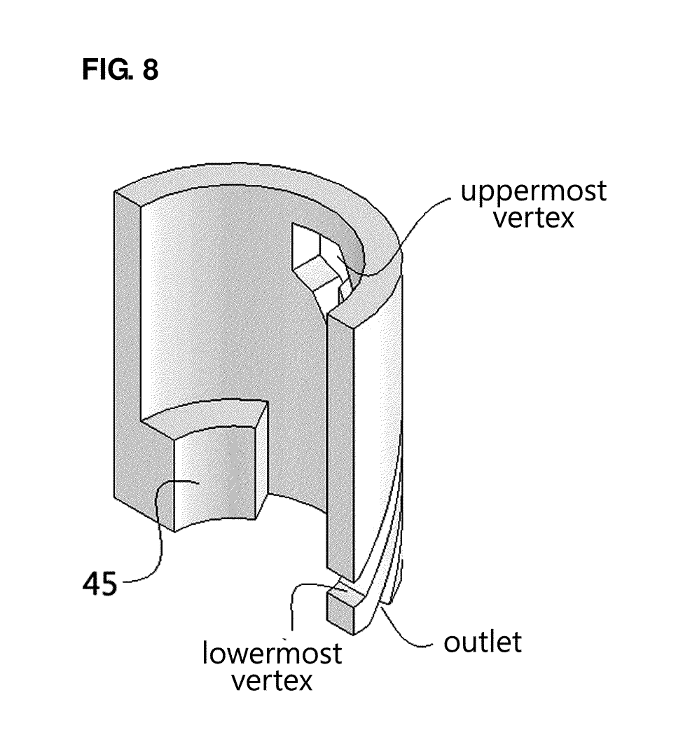

The rotation drive unit 40 includes a retaining step 45 circumferentially protruding inwardly from one side of the inner peripheral surface thereof so that the stop projection 21 of a descending lancet block is retained by the retaining step 45 to temporarily restrict the downward movement of the lancet block (see FIG. 8). As a result, the lancet housing 20 is temporarily stopped in its vertical movement and only the lancet 10 mounted inside the lancet housing 20 is moved vertically. In this case, if the lancet housing 20 is additionally provided with the re-use prevention member 23, there occurs a deformation in the re-use prevention member 23.

In the present invention, the cylindrical housing 50 includes a plurality of support barrier walls 54 formed protrudingly outwardly from the inner peripheral surface thereof so as to receive the support jaw 43 of the rotation drive units 40 in a space defined between the support barrier walls so that the rotation drive unit is mounted within the cylindrical housing in such a fashion that the longitudinal movement thereof is restricted and the rotation thereof is enabled. The lancet-receiving member 30 is fixedly coupled to the cylindrical housing 50. The load button 60 is fittingly inserted into an upper end of the housing 50 in such a fashion that the outer periphery of the load button 60 comes into close contact with the inner periphery of the housing 50 so that the load button 60 can be moved vertically. The cylindrical housing 50 includes a rotation-preventing groove 53 longitudinally formed on the inner peripheral surface thereof so as to restrict the rotation of the load button 60.

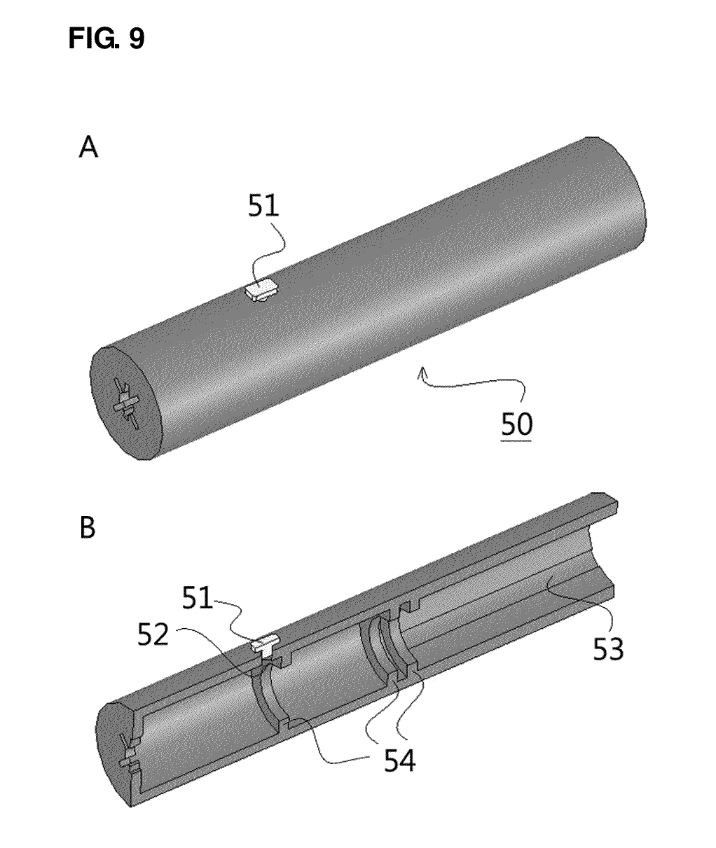

Also, the cylindrical housing includes a through-hole 52 formed thereon to correspond to a load position of the load-maintaining lug 41 of the rotation drive unit 40, and a drive button 51 fitted into the through-hole 52 so as to elastically apply a pressure to the load-maintaining lug 41 when the drive button is pressed (see FIG. 9).

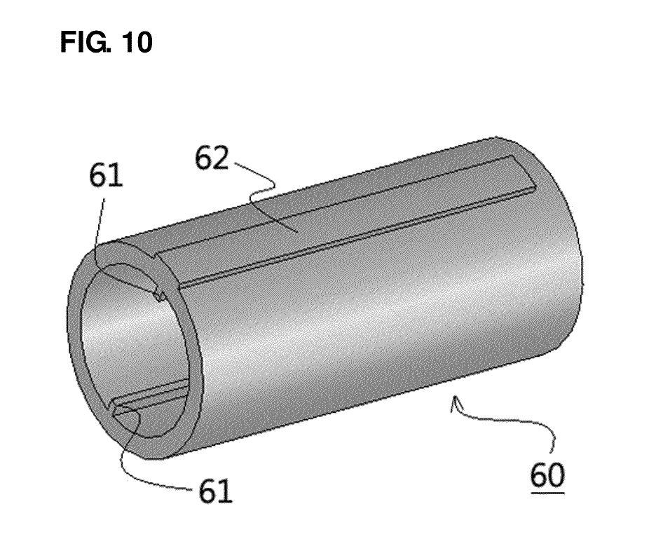

In the present invention, the load button 60 is fittingly inserted between the upper end of the housing 50 and the upper end of the rotation drive unit 40 so that the load button can move only in the longitudinal direction thereof. The load button 60 includes a rotation-preventing protrusion 62 longitudinally formed on the outer peripheral surface thereof to correspond to a position of the rotation-preventing groove 53 of the cylindrical housing 50, and a pair of opposed rotation guide ribs 61 longitudinally formed on the inner peripheral surface thereof to correspond to the rotation guide slot 44 of the rotation drive unit 40 so that the rotation guide ribs 61 push the rotation drive unit 40 while sliding along the rotation guide slot 44 of the rotation drive unit 40 upon the longitudinal movement of the load button (see FIG. 10).

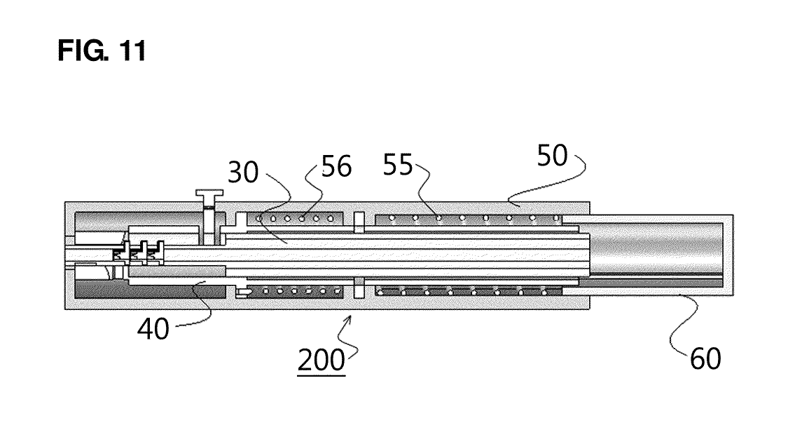

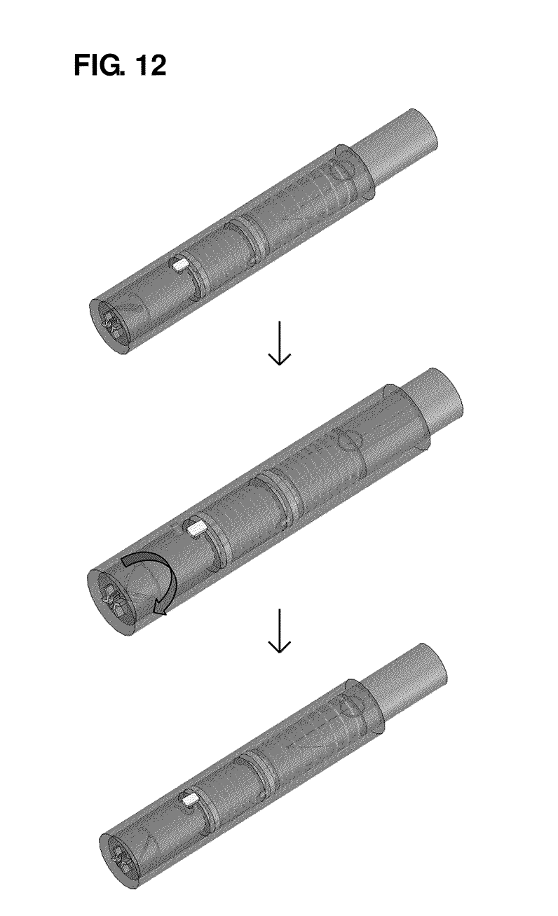

In the present invention, a vertical return spring 55 is interposed between one of the support barrier walls 54 of the housing 50 and an open end of the load button 60 so as to return the load button 60 pushed downwardly by an external force to it is original position. A rotational return spring 56 is interposed between another support barrier wall 54 of the housing 50 and one of the support jaws 43 of the rotation drive unit 40 in such a fashion as to be fixedly secured at one end to the housing 50 and at other end to the rotation drive unit 40 so as to return the rotation drive unit 40 rotated by means of an external force to its original position before the rotation (see FIGS. 11 and 12).

The skin penetration depth of the lancet block needle may be adjusted by making the length of the needle 13 different or by configuring a cap for adjusting the skin penetration depth of the lancet block like a conventional lancet device (not shown).

In the drawings, a structure is illustrated in which the rotation-preventing protrusion 62 is longitudinally formed on a side of the outer peripheral surface thereof for prevention of the rotation of the load button 60 and the rotation-preventing groove 53 corresponding to the rotation-preventing protrusion 62 is longitudinally formed on the inner peripheral surface of the cylindrical housing 50. Also, a structure is illustrated in which the spiral rotation guide slot 44 is formed on the upper end portion thereof for rotation of the rotation drive unit 40 in response to the downward movement of the load button 60 and the rotation guide ribs 61 corresponding to the spiral rotation guide slot 44 are longitudinally formed on the inner peripheral surface of the load button 60. However, it should be noted that the relative positions of corresponding elements in such structures can be changed as long as the same operating effects can be obtained. In other words, in the lancet activating device according to the present invention, the rotation-preventing groove 53 and the rotation-preventing protrusion 62 are respectively formed on a side of the peripheries of a portion where the housing 50 and the load button 60 abut against each other so as to prevent rotation of the load button 60. Also, the spiral rotation guide slot 44 and the rotation guide rib 61 are respectively formed on the peripheries of a portion where the rotation drive unit 40 and the load button 60 abut against each other so as to allow the rotation drive unit 40 to rotate in response to the downward movement of the load button 60.

Hereinafter, an operation process of the lancet activating device 200 according to the present invention as constructed above will be described with reference to FIGS. 12 to 14.

The terms “forward rotation” and “reverse rotation”, as used herein, refer to rotations in the opposite directions. The rotation of the rotation drive unit 40 by an external force is referred to as “forward rotation”, and the returning rotation of the rotation drive unit 40 by an elastic restoring force of the rotational return spring is referred to as “reverse rotation”.

The lancet activating device 200 according to the present invention is configured to have a cross-sectional structure as shown in FIG. 11. The lancet-receiving member 30 is fixedly coupled to the housing 50, the rotation drive unit is interposed between the housing 50 and the lancet-receiving member 30 such that it can perform the forward and reverse rotation relatively with respect to the housing 50 and the lancet-receiving member 30. When the load button 60 is moved downwardly by an external force and then the external force is released, the load button 60 returns to its original position before its downward movement. When the load button 60 is moved downwardly by the external force, the rotation drive unit 40 performs the forward rotation by a predetermined angle through the interaction between the rotation guide rib 61 of the load button 60 and the rotation guide slot 44 of the rotation drive unit 40 (see FIG. 12). When the rotation drive unit 40 performs the forward rotation, it is stopped in its rotation while the elastic load-maintaining lug 41 is retained in the through-hole 52 of the housing 50 and the rotation drive unit 40 possesses elasticity enabling it to return to its original position before its rotation by the rotational return spring (loaded state). Then, when an external force is exerted on the drive button 51 of the housing 50, the load-maintaining lug 41 is pushed from the through-hole 52 to cause the rotation drive unit 40 to perform the reverse rotation to return to its original position before its rotation by the elastic restoring force of the rotational return spring (returned state). This returned state can be considered to be a normal state since there is no external force exerted.

One or more lancet blocks are vertically mounted in the lancet-receiving member from the top to the bottom. The mounted lancet blocks may be sequentially moved downwardly by gravity or a means such as a cap mounted with a spring capable of downwardly pushing the lancet blocks mounted inside the lancet-receiving member. When the above operation process is performed in a state where the lancet blocks are mounted inside the lancet-receiving member, the lancet needle 13 pierces the skin of a patient while moving in the direction of downwards (for its skin penetration) and upwards (for its separation from the skin) between the loaded state and the returned state, which is called a “piercing process”.

In the normal state (i.e., returned state) where no external force is applied to the assembled lancet activating device, the uppermost vertex of the cam projection guide slot 42 of the rotation drive unit 40 is positioned just above the cam projection exposure slot opening 32 of the lancet-receiving member. Also, in the loaded state where an external force is applied to the load button 60 to cause the load-maintaining lug 41 of the rotation drive unit 40 to be retained in the through-hole 52 of the housing 50, the inlet of the cam projection guide slot 42 is positioned just above the cam projection exposure slot opening 32 of the lancet-receiving member.

In the meantime, the cam projection 12 of a single lancet block is always fittingly inserted into both the cam projection exposure slot opening 32 of the lancet-receiving member 30 and the cam projection guide slot 42 of the rotation drive unit 40 at a lower end portion where the cam projection exposure slot opening 32 and the cam projection guide slot 42 are positioned. Thus, when the lancet block is mounted inside the lancet-receiving member 30, the inlet of the cam projection guide slot 42 is positioned just above the cam projection exposure slot opening 32 of the lancet-receiving member in the loaded state, and thus cam projection 12 of the lancet block at the lowermost position is retained in the inlet of the cam projection guide slot 42. Since the lancet-receiving member is fixedly secured to the housing 50, the loaded lancet block is vertically moved downwardly by a vertical distance of the advancement guide section of the cam projection guide slot 42 in response to the reverse rotation of the rotation drive unit 40 for skin penetration of the needle, is then vertically re-moved upwardly by a vertical distance of the retraction guide section of the cam projection guide slot 42 for separation of the needle from the skin, and then goes over the uppermost vertex of the ejection guide section via the retraction guide section. In this case, in order to prevent the used lancet block completely moved to the ejection guide section from returning to the retraction guide section, preferably, an inclined wall defined between a guide slot section running from the lowermost vertex to the uppermost vertex and a guide slot section running from the uppermost vertex to the outlet is made of an elastically deformable material, and a width of a guide slot section adjacent to the uppermost vertex is formed smaller than the outer diameter of the cam projection 12.

In the returned state, the cam projection of the used lancet block is positioned at the uppermost vertex of the ejection guide section. Subsequently, in the process where the state is switched from the returned state to the loaded state by applying an external force to the load button, or if the state progresses until immediately before the loaded state, the rotation drive unit 40 performs the forward rotation and the cam projection of the used lancet block positioned at the uppermost vertex is moved downwardly along the ejection guide section so as to be finally ejected externally through the outlet.

This process will be described hereinafter in more detail.

When the load button 60 is pressed in a state where the lancet blocks is mounted inside the lancet-receiving member, the rotation guide rib 61 presses an inclined surface of the rotation guide slot 44 to cause the rotation drive unit 40 to perform the forward rotation so that the load-maintaining lug is retained in the through-hole 52 just below the drive button 51 to maintain the loaded state. Then, the load button returns to its original position by the vertical return spring and one of the lancet blocks mounted inside the lancet-receiving member is positioned at the inlet of the cam projection guide slot 42. In this loaded state, when the drive button 51 is pressed, the load-maintaining lug 51 escapes from the through-hole 52 to cause the rotation drive unit 40 to perform the reverse rotation to rapidly return to its original position by the rotational return spring. In this case, since the cam projection 12 of the lancet block is moved along the cam projection guide slot 42, the needle 13 of the lancet block is moved downwardly for skin penetration, and then is immediately moved upwardly along the retraction guide section for its separation from the skin. The cam projection of the used lancet block is moved along the ejection guide section in a next loading process or in the process of pressing the load button 60 by half, so that the used lancet block is ejected externally through an opening formed at the lower distal end of the lancet activating device.

In the meantime, when the cam projection 12 passes through an intermediate portion of the advancement guide section of the cam projection guide slot 42, the retaining step 45 formed on the inner peripheral surface of the rotation drive unit abuts against the stop projection 21 of the lancet housing 20 to stop the vertical movement of the lancet housing 20 and permit only the vertical movement of the lancet 10. Thereafter, when the cam projection 12 passes through the ejection guide section, the retaining step 45 is placed at a position that does not allow abutting against the stop projection 21. That is, in the case where the retaining step 45 is formed just below a position of the stop projection 21 located when the cam projection 12 is moved downwardly along the advancement guide section, when the cam projection 12 is moved downwardly along the ejection guide section, the rotation drive unit 40 rotates, so that the stop projection 21 is not retained by the retaining step 45 any more.

In the meantime, when the lancet housing 20 passes through the retraction guide section, the lancet 10 returns to its original position within the housing 50 by means of its own elasticity or a spring for returning the lancet 10 so that the needle 13 is not exposed externally. Thus, the used lancet 10 ejected in such a pattern can prevent any injury by a tip of the needle 13 and bacterial infection due to the injury since its needle 13 is not exposed externally.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A lancet block for blooding sampling comprising:
(a) a lancet comprising a body, a needle formed at the bottom of the body, and a cam projection of a predetermined length formed at a side of the body; and
(b) a lancet housing receiving the lancet in such a fashion that the lancet can be vertically moved relatively with respect to the lancet housing, and including a stop projection formed at an outer side thereof,
wherein a top wall of the lancet housing and a side wall of the lancet housing corresponding to the cam projection of the lancet are formed of a thin membrane made of a material capable of being easily disrupted or a material capable of being expanded elastically upon relative downward movement of the lancet with respect to the lancet housing, and a bottom wall of the lancet housing is formed of a thin membrane made of a material capable of being easily disrupted by a tip of the needle of the lancet.

2. The lancet block for blooding sampling according to claim 1, further comprising a spring mounted within the lancet housing so as to return the lancet downwardly moved relatively with respect to the lancet housing to its original position.

3. The lancet block for blooding sampling according to claim 1, wherein the lancet housing further comprises a re-use prevention member formed on the bottom of the stop projection thereof so as to allow the re-use prevention member to be disrupted or deformed by a pressure.

4. The lancet block for blooding sampling according to claim 1, wherein the lancet housing is formed in the shape of a polygonal or cylindrical box.

5. The lancet block for blooding sampling according to claim 1, wherein the lancet is made of a biocompatible thermoplastic resin material.

6. The lancet block for blooding sampling according to claim 2, wherein the lancet housing further comprises a re-use prevention member formed on the bottom of the stop projection thereof so as to allow the re-use prevention member to be disrupted or deformed by a pressure.

7. The lancet block for blooding sampling according to claim 2, wherein the lancet housing is formed in the shape of a polygonal or cylindrical box.

8. The lancet block for blooding sampling according to claim 2, wherein the lancet is made of a biocompatible thermoplastic resin material.

9. A lancet activating device adapted to activate the lancet block according to claim 1, the lancet activating device comprising a lancet-receiving member, a rotation drive unit, a housing, a load button, a vertical return spring, and a rotational return spring,
(a) wherein the lancet-receiving member includes an upper portion and a lower portion extending by a predetermined length from a lower distal end thereof, the lower portion having an outer diameter smaller than that of the upper portion,
wherein the lancet-receiving member includes a lancet block movement passageway penetratingly formed longitudinally therein so that one or more lancet blocks can be fittingly inserted into the lancet block movement passageway in the longitudinal direction so as to be vertically and longitudinally moved but restricted in their rotation along the lancet block movement passageway and a cam projection exposure slot opening formed longitudinally on the periphery of the lower portion of the lancet-receiving member so as to allow a cam projection of a lancet block to be exposed externally therethrough, and
wherein the lancet-receiving member is fittingly inserted into the rotation drive unit in such a fashion that an outer periphery of the lancet-receiving member comes into close contact with an inner periphery of the rotation drive unit so that the lancet-receiving member can rotate relatively with respect to the rotation drive unit within the rotation drive unit, and is fixedly coupled to the housing;
(b) wherein the rotation drive unit is configured such that the lancet-receiving member is fittingly inserted into the rotation drive unit in such a fashion that the outer periphery of the lancet-receiving member comes into close contact with the inner periphery of the rotation drive unit,
wherein the load button is fittingly inserted into an upper end of the rotation drive unit in such a fashion that the inner periphery of the load button comes into close contact with the outer periphery of the rotation drive unit so that the load button can move vertically in the longitudinal direction thereof, and
wherein the rotation drive unit has a cam projection guide slot formed on the periphery of a lower end portion thereof to correspond to the cam projection exposure slot opening of the lancet-receiving member so that the cam projection of the lancet block protruded outwardly through the cam projection exposure slot opening is fittingly inserted into the cam projection guide slot so as to allow the lancet to be guided to move in the direction of downwards (for its skin penetration), upwards (for its separation from the skin) and downwards (for its ejection) in this order, a retaining step circumferentially formed on one side of the inner peripheral surface thereof so as to temporarily restrict the downward movement of the stop projection of the lancet block, an elastic load-maintaining lug formed on the outer peripheral surface of a lower portion thereof in such a fashion as to be spaced remote from the cam projection guide slot, and at least one support jaw formed on the outer peripheral surface thereof so as to be received in one or more support barrier walls of the housing so that the longitudinal movement of the rotation drive unit is restricted and the rotation of the rotation drive unit is enabled in response to the vertical movement of the load button;
(c) wherein the housing includes one or more support barrier walls formed on the inner peripheral surface thereof so as to receive the support jaw of the rotation drive units in a space defined between the support barrier walls so that the rotation drive unit is mounted within the housing in such a fashion that the longitudinal movement of the rotation drive units is restricted and the rotation of the rotation drive units is enabled,
wherein the housing is fixedly coupled with the lancet-receiving member,
wherein the load button is fittingly inserted into an upper end of the housing in such a fashion that the outer periphery of the load button comes into close contact with the inner periphery of the housing so that the load button is restricted in the rotation thereof and can move vertically in the longitudinal direction thereof, and
wherein the housing has a through-hole formed on the outer peripheral surface thereof to correspond to the load position of the load-maintaining lug of the rotation drive unit, and a drive button 51 fittingly mounted into the through-hole so as to elastically apply a pressure to the load-maintaining lug when the drive button is pressed;
(d) wherein the load button is fittingly inserted between the upper end of the housing and the upper end of the rotation drive unit so that the load button can move only in the longitudinal direction thereof and allows the rotation drive unit to rotate upon the longitudinal movement thereof; and
(e) wherein the vertical return spring is interposed between a support barrier wall of the housing and an open end of the load button so as to return the load button pushed downwardly by an external force to it is original position, and the rotational return spring is interposed between a support barrier wall of the housing and the support jaw of the rotation drive unit in such a fashion as to be fixedly secured at one end to the housing and at other end to the rotation drive unit so as to return the rotation drive unit rotated by means of an external force to its original position before the rotation.

10. The lancet activating device according to claim 9, wherein the rotation-preventing groove and the rotation-preventing protrusion are respectively formed on a side of the peripheries of a portion where the housing and the load button abut against each other so as to prevent rotation of the load button, and
wherein the spiral rotation guide slot and the rotation guide rib are respectively formed on the peripheries of a portion where the rotation drive unit and the load button abut against each other so as to allow the rotation drive unit to rotate in response to the downward movement of the load button.

11. The lancet activating device according to claim 9, wherein the horizontal cross-sectional shape of the lancet block movement passageway of the lancet-receiving member corresponds to the horizontal cross-sectional shape of the lancet block.

12. The lancet activating device according to claim 9, wherein an inlet of the cam projection guide slot of the rotation drive unit is formed at a position corresponding to an upper end of the cam projection exposure slot opening of the lancet-receiving member, and an outlet of the cam projection guide slot is opened toward a lower end of the housing, and
wherein a guide slot section running from the inlet via a lowermost vertex to a uppermost vertex is formed in a “V” shape, and a guide slot section running from the uppermost vertex to the outlet is formed in an oblique shape.

13. The lancet activating device according to claim 12, wherein in the cam projection guide slot, an inclined wall defined between a guide slot section running from the lowermost vertex to the uppermost vertex and a guide slot section running from the uppermost vertex to the outlet is made of an elastically deformable material, and a width of a guide slot section adjacent to the uppermost vertex is formed smaller than the outer diameter of the cam projection.

14. The lancet activating device according to claim 9, wherein the lancet-receiving member further comprises a cap mounted with a spring provided at a distal end of the upper portion thereof so that the spring downwardly pushes the lancet blocks mounted inside the lancet-receiving member.

15. The lancet activating device according to claim 9, wherein the housing further comprises a skin penetration length-adjusting cap provided at a lower distal end thereof so as to adjust the protruding length of the needle.

16. The lancet activating device according to claim 10, wherein the horizontal cross-sectional shape of the lancet block movement passageway of the lancet-receiving member corresponds to the horizontal cross-sectional shape of the lancet block.

17. The lancet activating device according to claim 10, wherein an inlet of the cam projection guide slot of the rotation drive unit is formed at a position corresponding to an upper end of the cam projection exposure slot opening of the lancet-receiving member, and an outlet of the cam projection guide slot is opened toward a lower end of the housing, and
wherein a guide slot section running from the inlet via a lowermost vertex to a uppermost vertex is formed in a “V” shape, and a guide slot section running from the uppermost vertex to the outlet is formed in an oblique shape.

18. The lancet activating device according to claim 17, wherein in the cam projection guide slot, an inclined wall defined between a guide slot section running from the lowermost vertex to the uppermost vertex and a guide slot section running from the uppermost vertex to the outlet is made of an elastically deformable material, and a width of a guide slot section adjacent to the uppermost vertex is formed smaller than the outer diameter of the cam projection.

19. The lancet activating device according to claim 10, wherein the lancet-receiving member further comprises a cap mounted with a spring provided at a distal end of the upper portion thereof so that the spring downwardly pushes the lancet blocks mounted inside the lancet-receiving member.

20. The lancet activating device according to claim 10, wherein the housing further comprises a skin penetration length-adjusting cap provided at a lower distal end thereof so as to adjust the protruding length of the needle.
